(19) United States
(12) Patent Application Publication
(10) Pub. No.: US 2017/0202632 A1
(43) Pub. Date: Jul. 20, 2017

(54) CORDLESS EXAMINATION LAMP

(71) Applicant: Papastork Lighting, LLC, Wilmette, IL (US)

(72) Inventor: Ronald Miller, Wilmette, IL (US)

(21) Appl. No.: 15/407,884

(22) Filed: Jan. 17, 2017

Related U.S. Application Data
(60) Provisional application No. 62/279,071, filed on Jan. 15, 2016.

(51) Int. Cl. A61B 90/00

(52) U.S. Cl. CPC A61B 90/36

(57) ABSTRACT

A cordless examination lamp which includes a base with wheels or casters. The base has a body securely connected. A flexible neck portion connects to the body and a light source is connected to that flexible neck portion. The cordless examination lamp is powered by a rechargeable power source.

### RELATED APPLICATIONS

This application claims priority to U.S. Provisional Application No. 62/279,071 titled “CORDLESS EXAMINATION LAMP” which was filed on Jan. 15, 2016. The '071 application is hereby incorporated by reference.

### TECHNICAL FIELD OF THE INVENTION

The present invention relates to lamps used for medical examinations. Particularly, the application relates to cordless examination lamps for use in medical examinations.

### BACKGROUND OF THE INVENTION

It can be critically important for a physician to have good lighting during a medical examination or procedure. The use of precisely-focused natural white light has been found to provide the most useful lighting aid. However, such lighting is typically provided either by a stationary lamp above an examination/operating table or by a movable floor lamp plugged into a wall outlet.

The stationary lamp is not particularly useful when light is needed, for example, from a lower angle or away from the table over which it is positioned. The stationary examination lamp does not have flexibility or mobility needed by examining physicians.

The movable floor lamp also has drawbacks. In a busy examination room a lamp cord becomes a tripping hazard for patients and staff Further, the cord anchors the lamp to the outlet and limits the range of movement. Relocation of the lamp plug to a more convenient outlet adds delay to the examination process, which is undesirable, or may not be possible due to outlet unavailability.

These and other problems are addressed by the present device to provide a system with numerous advantages in operation and effectiveness.

### SUMMARY OF THE INVENTION

There is disclosed herein a cordless examination lamp which avoids the disadvantages of prior examination lamps while affording additional structural and operating advantages.

Generally speaking, the cordless examination lamp is comprised of a base, a body, a flexible neck portion, a light source and a power source located on one of either the base, body or neck portion.

In specific embodiments of the cordless examination lamp, the light source is preferably comprised of an LED. The light source is attached to the flexible neck portion, preferably at a free end. The flexible neck portion is moveable, bendable, and possibly twistable to allow the cordless examination light to illuminate areas at a variety of angles, including lower angled areas and areas away from an examination table. The light source is preferably detachable from the flexible neck portion. In an embodiment, the light source is powered by alkaline batteries which can be changed once the light source is detached from the flexible neck portion.

The body may be extendable and retractable to allow illumination from the cordless examination lamp to occur at various heights. The body is attached to the base that allows for the cordless examination lamp to be easily moveable and secured once placed in a desired location.

In specific embodiments, the base preferably has five legs for greater stability with each leg having a caster thereon. Alternatively, at least one leg may have a mounting device which allows the base to be appropriately secured to a surface or to the ground. In other embodiments, the legs might include wheels, allowing the cordless examination lamp to be easily moved during an examination.

In specific embodiments, the power source is comprised of a battery pack. Preferably, the battery pack is attached to the base to power the cordless examination lamp. A preferred embodiment of the battery pack includes a rechargeable LI-ion battery. Alternatively, alkaline batteries can be used in the light source to power the cordless examination lamp.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 is a exploded view of an embodiment of the examination lamp light source in accordance with the present disclosure;

FIG. 2 is a close-up side view of an embodiment of the examination lamp light source in accordance with the present disclosure;

FIG. 3 is a close up rear view of an embodiment of the examination lamp light source in accordance with the present disclosure;

FIG. 4 is a close view of an embodiment of the examination lamp base having a rechargeable power source in accordance with the present disclosure; and

FIG. 5 is a full view of an embodiment of the examination lamp.

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

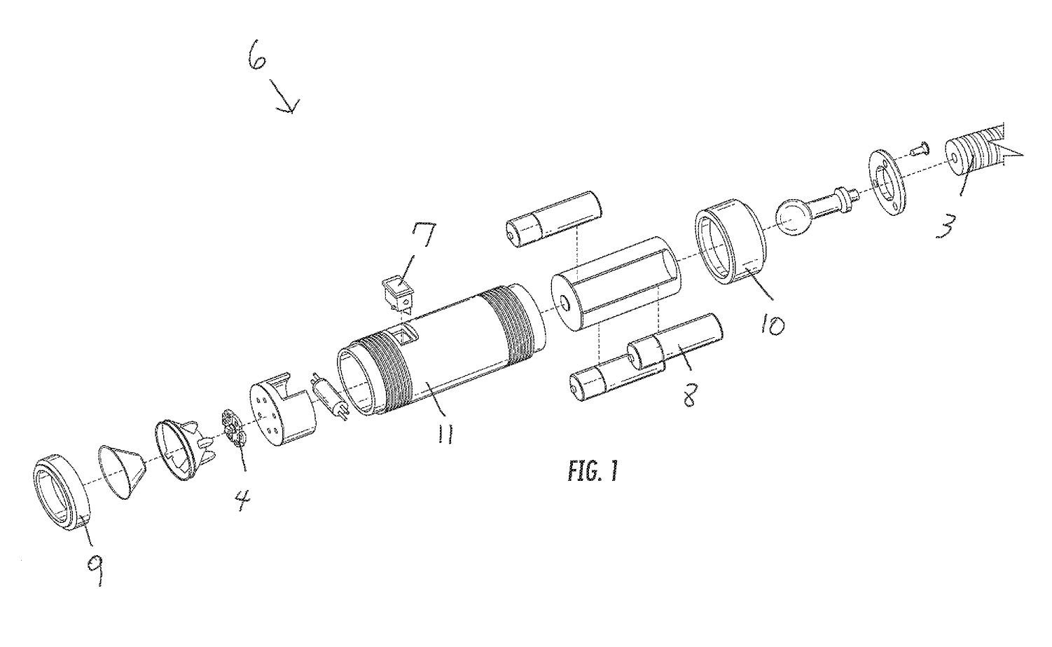

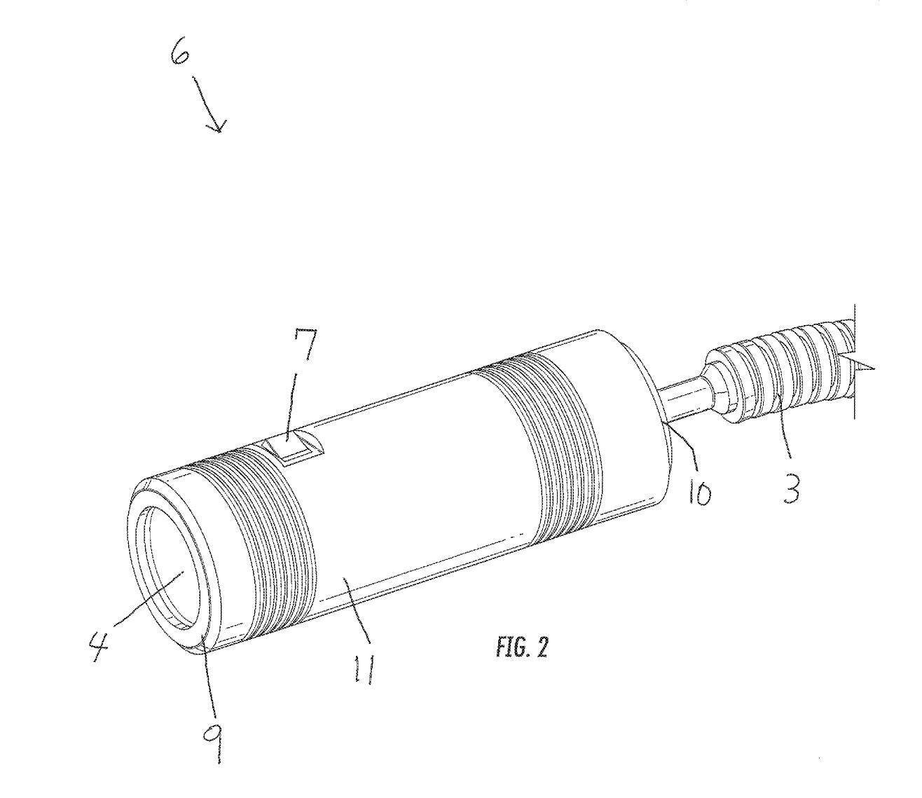

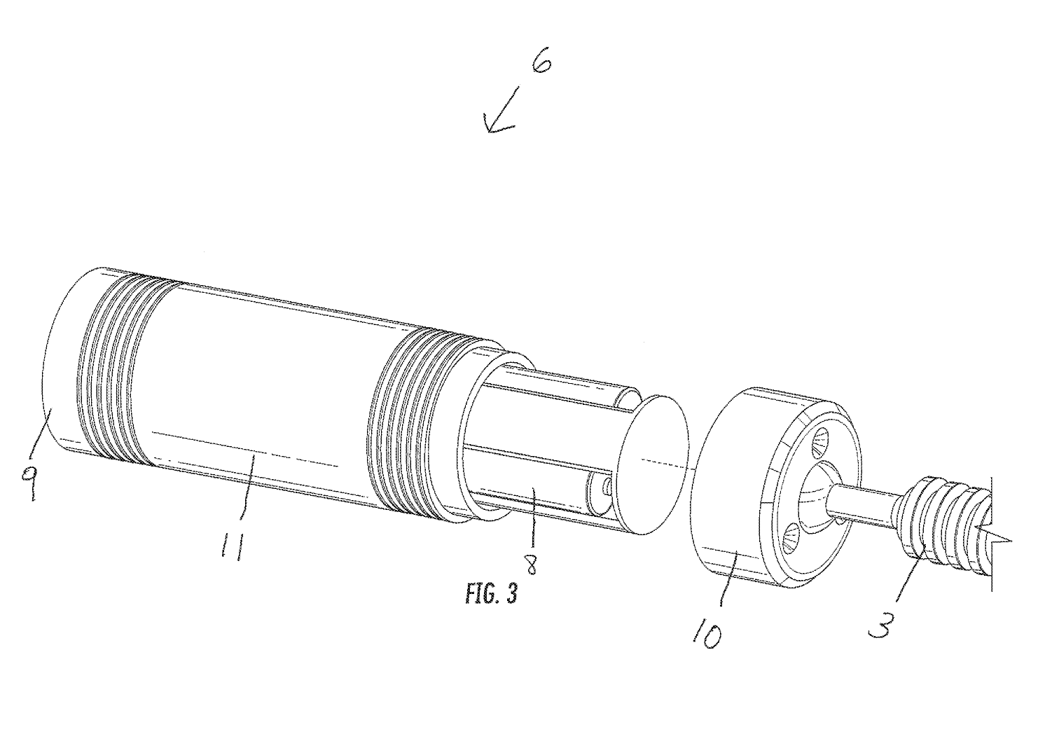

Referring to FIGS. 1-5, there is illustrated and described embodiments of the cordless examination lamp, generally referenced by the number 1, including the various components of the lamp 1. Generally speaking, the lamp 1 is comprised of a base 2, a body 5, a flexible neck portion 3, a light source 4 and a power source 8 located on one of the base 2, the body 5 or the neck portion 3.

An embodiment of the light source assembly 6 is shown in FIGS. 1, 2 and 3. The illustrated assembly 6 is comprised of a housing 11 which houses the light source 4 (preferably, at least one LED) and the power source (e.g., batteries) 8, and connects to the neck portion 3 via connecting end 10. An LED cover 9 protects the LEDs and a power switch 7 is mounted on a surface of the housing 11 for easy access. The connecting end 10 is preferably comprised of a “ball-and-socket” joint which allows maximum movement of the illumination from the light source 4. This configuration also allows complete control by an examining physician using only one hand. The light source 4 can be turned on and off at the power switch 7, and adjusted for height and angle from the assembly 6. Even the power source 8 is located within the assembly for ease of changing.

In a specific embodiment, the connecting end 10 of the assembly 6 may allow detachment of the assembly 6 from the flexible neck portion 3. This feature may allow a physician to bring the light very close to a particular area of a patient for examination without the rest of the examination lamp being in the way.

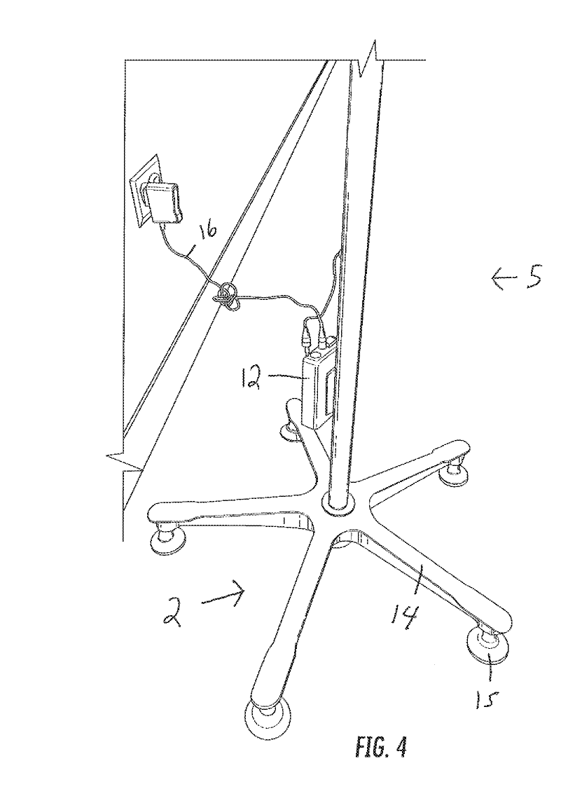

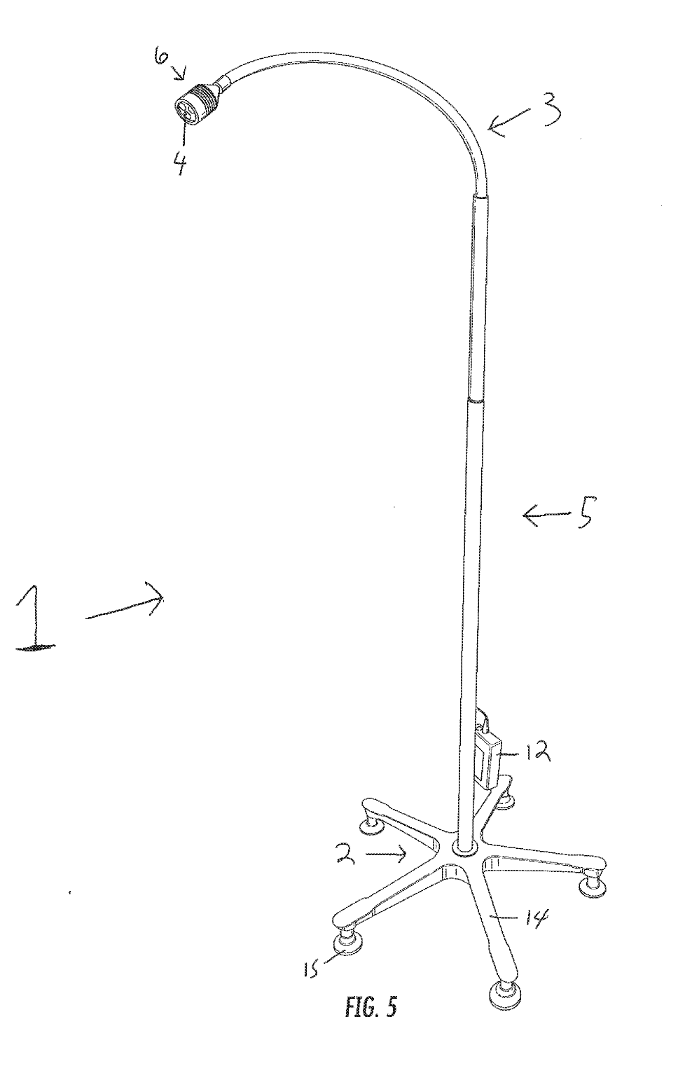

Referring to FIGS. 4 and 5, the supporting features of the cordless examination lamp 1 can be more readily appreciated. Supporting the light source assembly 6, is the flexible neck portion 3, via the connecting end 10, which is then connected to the body 5 of the lamp 1, and supported for movement by the base 2. Further, in the illustrated embodiment, a rechargeable power source 12 is positioned on the body 2. As an alternative to the light source assembly 6 power source 8, the lamp 1 may include a rechargeable battery (not shown) with a charging cord 16 for charging when the lamp 1 is not in use. Of course, this rechargeable feature may also be used in the light source assembly 6, as desired.

As can also be seen in FIG. 4, the base is comprised of a plurality of legs 14 for stability. The legs 14 preferably include means to allow the lamp 1 to be moved about without carrying. For example, casters or wheels compatible with an examination floor may be suitable. Alternatively, especially where carpet is used, sliders (not shown) can be used on the ends of the legs to allow the base to slide in any direction when pulled. Another alternative embodiment might include a mounting device 15 on a leg of the base 2, or on the base itself. Such a device might allow temporary attachment to an examination table, for example.

Finally, as illustrated in FIG. 5, the body 5 of the lamp may allow for telescoping to raise and lower the light source 4. The length and number of telescoping segments will determine the amount of lowering and raising of the light source 4 is possible. Other means for raising and lowering the light source 4 may also be used.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

1. An examination lamp comprising:
a base;
a body secured to the base; and
a flexible neck portion extending from the body;
a light source at an end of the flexible neck portion; and
a rechargeable power source attached to one of either the base, body or neck.

2. The examination lamp of claim 1, wherein the base comprises wheels or casters for facilitating movement of the lamp.

3. The examination lamp of claim 1, wherein the base comprises a mounting device.

4. The examination lamp of claim 1, wherein the light source comprises at least one LED.

5. The examination lamp of claim 1, wherein the rechargeable power source comprises a rechargeable Li-ion battery.

6. The examination lamp of claim 1, wherein the light source comprises a battery.

7. The examination lamp of claim 1, wherein the light source is detachable from the flexible neck portion.

8. The examination lamp of claim 1, wherein the light source has a connecting end.

9. The examination lamp of claim 8, wherein the connecting end comprises a ball and socket joint.

10. The examination lamp of claim 1, wherein the flexible neck portion allows the light source to be moved from the light source itself.

11. The examination lamp of claim 1, wherein the body is extendable and retractable.

12. The examination lamp of claim 11, wherein the body is comprised of telescoping segments.

13. The examination lamp of claim 1, wherein the LED is protected by a LED cover.

14. An examination lamp comprising:
a base having surface contacting members which facilitate movement of the base on a surface;
an adjustable body secured to the base and comprised of a plurality of telescoping segments;
a flexible neck portion extending from the body; and
a light source assembly attached to an end of the flexible neck portion, wherein the light source assembly comprises:
a housing attached to the flexible neck portion via a ball and socket joint to allow adjustment of the light source assembly;
a least one LED positioned with the housing; and
a rechargeable power source positioned within the housing to power the at least one LED.
